(19) United States
(12) Patent Application Publication
(10) Pub. No.: US 2004/0197295 A1
(43) Pub. Date: Oct. 7, 2004

(54) Foamable preparations

(75) Inventors: Heidi Riedel, Schenefeld (DE); Rainer Kropke, Schenefeld (DE); Andreas Bleckmann, Hamburg (DE); Ilka Oelrichs, Tornesch (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/760,086

(22) Filed: Jan. 16, 2004

Related U.S. Application Data
(63) Continuation of application No. PCT/EP02/07907, filed on Jul. 16, 2002.

(30) Foreign Application Priority Data
Jul. 17, 2001 (DE) 101 34 786.3

(51) Int. Cl. A61K 7/075; A61K 7/08

(52) U.S. Cl. 424/70.22

(57) ABSTRACT

The invention is a foamable cosmetic or dermatological preparation comprising (I) an emulsifier system comprising (A) at least one emulsifier A selected from the group consisting of wholly neutralized, partially neutralized and unneutralized, branched and unbranched, saturated and unsaturated fatty acids having a chain length of from 10 to 40 carbon atoms, (B) at least one emulsifier B selected from the group consisting of polyethoxylated fatty acid esters having a chain length of from 10 to 40 carbon atoms and a degree of ethoxylation of from 5 to 100, and (C) at least one coemulsifier C selected from the group consisting of saturated and unsaturated, branched and unbranched fatty alcohols having a chain length of from 10 to 40 carbon atoms; and (II) up to 50% by weight—based on the total weight of the foamable preparation—of a lipid phase which comprises one or more lipids selected from the group consisting of silicone oils and silicone waxes.

### CROSS-REFERENCE TO RELATED APPLICATIONS

This is a continuation application of PCT/EP02/07907, filed Jul. 16, 2002, which is incorporated herein by reference in its entirety, and also claims the benefit of German Priority Application No. 101 34 786.3, filed Jul. 17, 2001.

### FIELD OF THE INVENTION

The present invention relates to foamable cosmetic and dermatological preparations, in particular to skincare foamable cosmetic and dermatological preparations.

### BACKGROUND OF THE INVENTION

Foams or foam-like preparations are a type of disperse system. By far the most important and best known disperse systems are emulsions. Emulsions are two- or multi-phase systems of two or more liquids which are insoluble or only slightly soluble in one another. The liquids (pure or as solutions) are present in an emulsion in a more or less fine distribution, which generally has only limited stability.

Foams are structures of gas-filled, spherical or polyhedral cells which are delimited by liquid, semi-liquid, high-viscosity or solid cell ribs. The cell ribs connected via points of intersection form a continuous framework. The foam lamellae stretch between the cell ribs (closed-cell foam). If the foam lamellae are disturbed or if they flow back into the cell rib at the end of foam formation, an open-cell foam is obtained. Foams too—like emulsions—are thermodynamically unstable since a reduction in the surface area leads to the production of surface energy. The stability and thus the existence of a foam is thus dependent on to what extent it is possible to prevent its self-destruction.

Cosmetic foams are usually dispersed systems of liquids and gases, where the liquid represents the dispersant and the gas represents the dispersed substance. Foams of low-viscosity liquids are temporarily stabilized by surface-active substances (surfactants, foam stabilizers). Because of their large internal surface area, such surfactant foams have a high adsorption capacity, which is utilized, for example, in cleaning and washing operations. Accordingly, cosmetic foams are used, in particular, in the fields of cleansing, for example as shaving foam, and of hair care.

To generate foam, gas is bubbled into suitable liquids, or foam formation is achieved by vigorously beating, shaking, spraying or stirring the liquid in the gas atmosphere in question, provided that the liquids comprise suitable surfactants or other interface-active substances (“foam formers”), which, apart from interfacial activity, also have a certain film-forming ability.

Cosmetic foams have the advantage over other cosmetic preparations of permitting a fine distribution of active ingredients on the skin. However, cosmetic foams can generally only be achieved using particular surfactants, which, moreover, are often not well tolerated by the skin.

A further disadvantage of the prior art is that such foams have only low stability, for which reason they usually collapse within approximately 24 hours. A requirement of cosmetic preparations, however, is that they have stability for years, as far as possible.

This problem is generally taken into account by the fact that the consumer produces the actual foam himself just before use using a suitable spray system for which purpose, for example, it is possible to use spray cans in which a liquefied pressurized gas serves as propellant gas. Upon opening the pressure valve, the propellant liquid mixture escapes through a fine nozzle, and the propellant evaporates, leaving behind a foam. Such systems according to the prior art develop, under the addition of propellant gas, exclusively aqueous-moist foams which rapidly break following application.

After-foaming cosmetic preparations are also known per se. They are firstly applied to the skin from an aerosol container in flowable form and, after a short delay, develop the actual foam only once they are on the skin under the effect of the after-foaming agent present, for example a shaving foam. After-foaming preparations are often in specific formulation forms, such as, for example, after-foaming shaving gels or the like.

### SUMMARY OF THE INVENTION

An object of the present invention was to enrich the prior art and to provide cosmetic or dermatological foamable preparations which do not have the disadvantages of the prior art.

German laid-open specification DE 197 54 659 discloses that carbon dioxide is a suitable active ingredient for stabilizing or increasing the epidermal ceramide synthesis rate, which may serve to enhance the permeability barrier, reduce the transepidermal water loss and increase the relative skin moisture. To treat the skin, the C₂ is, for example, dissolved in water, which is then used to rinse the skin. However, the prior art hitherto does not include any sort of cosmetic or dermatological bases in which a gaseous active ingredient could be incorporated in an adequate, i.e., effective, concentration.

It was thus a further object of the present invention to find cosmetic or dermatological bases into which effective amounts of gaseous active ingredients can be incorporated.

It was surprising and could not have been foreseen by the person skilled in the art that foamable cosmetic or dermatological preparations which comprise

I. an emulsifier system which consists of

A. at least one emulsifier A chosen from the group of wholly neutralized, partially neutralized or unneutralized branched and/or unbranched, saturated and/or unsaturated fatty acids having a chain length of from 10 to 40 carbon atoms,

B. at least one emulsifier B chosen from the group of polyethoxylated fatty acid esters having a chain length of from 10 to 40 carbon atoms and a degree of ethoxylation of from 5 to 100 and

C. at least one coemulsifier C chosen from the group of saturated and/or unsaturated, branched and/or unbranched fatty alcohols having a chain length of from 10 to 40 carbon atoms, and

II. up to 50% by weight—based on the total weight of the foamable preparation—of a lipid phase which comprises one or more lipids from the group of silicone oils and silicone waxes

overcome the disadvantages of the prior art.

For the purposes of the present invention, foamable is understood as meaning that the preparations according to the invention form foams when they are formed, i.e., when, for example, gas is bubbled into them or the preparations are (vigorously) beaten, shaken, sprayed or stirred into the gas atmosphere in question. In foams produced in this way, the gas bubbles may be present in (arbitrary) distribution in one (or more) liquid phase(s), where the foams do not necessarily have to have the appearance of a foam in macroscopic terms.

From foamable cosmetic or dermatological preparations according to the invention it is possible to prepare, by foaming, macroscopically visibly dispersed systems of gases dispersed in liquids. The foam character can, however, for example, be visible also only under a (light) microscope. Moreover, the foaming of the foamable preparations according to the invention is, particularly when the gas bubbles are too small to be recognized under a light microscope, also recognizable from a sharp increase in volume of the system.

The preparations according to the invention are entirely satisfactory preparations in every respect. It was particularly surprising that the foams produced from the foamable preparations according to the invention are extraordinarily stable, even in cases of an unusually high gas volume. Accordingly, for the purposes of the present invention they are very suitable for use as bases for product forms having diverse use purposes.

The preparations according to the invention and foams obtainable therefrom have very good sensory properties, such as, for example, distributability on the skin or the ability to be absorbed into the skin, and are, moreover, characterized by above-average skincare.

Finely bubbled, rich foams with excellent cosmetic elegance are obtainable from compositions according to the invention. In addition, preparations which are particularly well tolerated by the skin are obtainable from compositions according to the invention, it being possible for valuable ingredients to be distributed particularly well on the skin.

The invention further provides for the use of foamable cosmetic or dermatological preparations which comprise

I. an emulsifier system which consists of

A. at least one emulsifier A chosen from the group of wholly neutralized, partially neutralized or unneutralized branched and/or unbranched, saturated and/or unsaturated fatty acids having a chain length of from 10 to 40 carbon atoms,

B. at least one emulsifier B chosen from the group of polyethoxylated fatty acid esters having a chain length of from 10 to 40 carbon atoms and a degree of ethoxylation of from 5 to 100 and

C. at least one coemulsifier C chosen from the group of saturated and/or unsaturated, branched and/or unbranched fatty alcohols having a chain length of from 10 to 40 carbon atoms, and

II. up to 50% by weight—based on the total weight of the foamable preparation—of a lipid phase which comprises one or more lipids from the group of silicone oils and silicone waxes

as cosmetic or dermatological bases for gaseous active ingredients.

### DETAILED DESCRIPTION OF THE PREFERRED EMBODIMENTS

The emulsifier(s) A is/are preferably chosen from the group of fatty acids which have been wholly or partially neutralized with customary alkalis (such as, for example, sodium hydroxide and/or potassium hydroxide, sodium carbonate and/or potassium carbonate, and mono- and/or triethanolamine). Stearic acid and stearates, isostearic acid and isostearates, palmitic acid and palmitates, and myristic acid and myristates, for example, are particularly advantageous.

The emulsifier(s) B is/are preferably chosen from the following group: PEG-9 stearate, PEG-8 distearate, PEG-20 stearate, PEG-8 stearate, PEG-8 oleate, PEG-25 glyceryl trioleate, PEG-40 sorbitan lanolate, PEG-15 glyceryl ricinoleate, PEG-20 glyceryl stearate, PEG-20 glyceryl isostearate, PEG-20 glyceryl oleate, PEG-20 stearate, PEG-20 methylglucose sesquistearate, PEG-30 glyceryl isostearate, PEG-20 glyceryl laurate, PEG-30 stearate, PEG-30 glyceryl stearate, PEG-40 stearate, PEG-30 glyceryl laurate, PEG-50 stearate, PEG-100 stearate, PEG-150 laurate. Particularly advantageous are, for example, polylethoxylated stearic esters.

The coemulsifier(s) C is/are preferably chosen according to the invention from the following group: butyloctanol, butyidecanol, hexyloctanol, hexyldecanol, octyldodecanol, behenyl alcohol (C₂₂H₄₅OH), cetearyl alcohol [a mixture of cetyl alcohol (C₁₆H₃₃OH) and stearyl alcohol (C₁₈H₃₇OH)], lanolin alcohols (wool wax alcohols, which are the unsaponifiable alcohol fraction of wool wax which is obtained following the saponification of wool wax). Particular preference is given to cetyl alcohol and cetylstearyl alcohol.

It is advantageous according to the invention to choose the weight ratios of emulsifier A to emulsifier B to coemulsifier C (A:B:C) as a:b:c, where a, b and c, independently of one another, may be rational numbers from 1 to 5, preferably from 1 to 3. Particular preference is given to a weight ratio of approximately 1:1:1.

It is advantageous for the purposes of the present invention to choose the total amount of emulsifiers A and B and of coemulsifier C from the range from 1 to 20% by weight, advantageously from 2 to 15% by weight, in particular from 5 to 10% by weight, in each case based on the total weight of the formulation.

The foamable cosmetic or dermatological compositions according to the invention can, for example, be dispensed from aerosol containers and thereby be foamed. Aerosol containers according to the invention are spray devices with a filling of the liquid or slurry-like substances which are under the pressure of a propellant (pressurized gas or aerosol packings). Such containers can be fitted with valves of very diverse construction which permit removal of the contents in the form of a foam.

Suitable pressurized gas containers for the purposes of the present invention are primarily cylindrical vessels made of metal (aluminum, tinplate, contents <1000 ml), protected or shatter resistant glass or plastic (contents <220 ml) or shattering glass or plastic (contents <150 ml), in the choice of which compressive strength and breaking strength, corrosion resistance, ease of filling, or ease of sterilizing etc., but also aesthetic aspects, handlability, printing properties, etc., play a role. The maximum permissible operating pressure of spray cans made of metal at 50° C is 12 bars and the maximum fill volume at this temperature is about 90% of the total volume. For glass and plastic cans, the values for the operating pressure are lower and dependent on the size of the container and the propellant (whether liquefied, compressed or dissolved gas).

For the purposes of the present invention, cans made of tinplate, aluminum and glass are particularly advantageous. For reasons of corrosion protection, metal cans can be coated on the inside (silver- or gold-coated), for which purpose all standard commercial internal protective coatings are suitable. For the purposes of the present invention, preference is given to polyester, epoxyphenol and polyamide-imide coatings. Film laminates made of polyethylene (PE), polypropylene (PP) and/or polyethylene terephthalate (PET) on the inside of the cans are also advantageous, in particular for cans made of tinplate.

The pressurized gas containers are usually single-part or two-part, but in most cases three-part cylindrical, conical or differently shaped. If plastics are used as the spray container material, then these should be resistant to chemicals and the sterilization temperature, gas-tight, impact-resistant and stable to internal pressures in excess of 12 bars. In principle, polyacetals and polyamides are suitable for spray container purposes.

The internal construction of the spray cans and the valve construction are many and varied, depending on the intended use and the physical nature of the ingredient—e.g. whether it is in the form of a two-phase or three-phase system—and can be determined by the person skilled in the art by simple trial and error without inventive activity. For suitable variants, reference may be made to the “Aerosol Technologie Handbuch der Aerosol-Verpackung” [Aerosol Technology handbook of Aerosol Packaging] (Wolfgang Tauscher, Melcher Verlag GmbH Heidelberg/Munich, 1996).

Valves which are advantageous according to the invention can be designed with or without riser tube. The individual components from which valves according to the invention are usually constructed preferably consist of the following materials:

Disc: tinplate: uncoated, gold- or clear-coated, film-laminated (PE, PP or PET) aluminum: uncoated, silver- or gold-coated, different coating variants, Stoner-average

Seal: natural or synthetic elastomers or thermoplastic (sleeve gaskets, film-lined made of PE or PP) internal and external seals, e.g. made of perbunan, buna, neoprene, butyl, CLB, LDPE, viton, EPDM, chlorobutyl, bromobutyl and/or diverse compounds

Cone: PA, POM, brass and diverse special materials, standard bores (e.g.: 0.25 to 0.70 mm or 2×0.45 to 2×1.00 mm), various shaft diameters

Spring: metal, particularly preferably V2A, stainless steel; plastic and also elastomer

Casing: standard and impact VPH bores, RPT bores or slit for overhead applications materials: e.g. polyacetal, PA, PE, POM and the like

Riser tube: plastic (polymer resin), e.g. PE, PP, PA or polycarbonate

Advantageous spray heads for the purposes of the present invention are, for example, foaming heads for upright use (hold can vertically) or foam heads for overhead application using one or more channels.

Suitable propellants are the customary “classic” readily volatile, liquefied propellant gases, such as, for example, dimethyl ether (DME) and/or linear or branched-chain hydrocarbons with two to five carbon atoms (such as, in particular, ethane, propane, butane, isobutane and/or pentane), which can be used on their own or in a mixture with one another.

Compressed air, and also other gases which are under pressure, such as air, oxygen, nitrogen, hydrogen, helium, krypton, xenon, radon, argon, nitrous oxide (N₂O) and carbon dioxide (CO₂) are also advantageously to be used for the purposes of the present invention as propellant gases (either on their own or in any desired mixtures with one another).

The person skilled in the art is naturally aware that there are other propellant gases which are nontoxic per se and which would be suitable in principle for realizing the present invention in the form of aerosol preparations, but which nevertheless should be omitted due to an unacceptable impact on the environment or other accompanying circumstances, in particular halogenated (substituted by fluorine, chlorin e, bromine, iodine and/or astatine) hydrocarbons, such as, for example, fluorocarbons and chlorofluorocarbons (CFCs).

For the purposes of the present invention, said gases can in each case be used individually or in any desired mixtures with one another.

For the purposes of the present invention, the volume fraction of propellant gas is advantageously chosen from the range from 0.1 to 30% by volume, based on the total volume of filler material and propellant gas (corresponding to a volume fraction of from 70 to 99.9% by volume of filler material).

A particularly preferred propellant gas for the purposes of the present invention is carbon dioxide. Foams obtainable from preparations according to the invention which comprise carbon dioxide as one of the active ingredients are particularly advantageous.

Particularly advantageous, finely creamy and rich foams are obtainable when the preparations according to the invention are foamed using linear or branched-chain, halogenated or nonhalogenated hydrocarbons. Very particularly advantageous foams are obtainable by foaming the preparations according to the invention with carbon dioxide, oxygen, compressed air and/or nitrogen.

It may be advantageous, although it is not necessary, for the formulations according to the present invention to comprise further emulsifiers. Preference is given to using those emulsifiers which are suitable for the preparation of W/O emulsions, it being possible for these to be present either individually or else in any combinations with one another.

The further emulsifier(s) is/are advantageously chosen from the group which comprises the following compounds:

polyglyceryl-2 dipolyhydroxystearate, PEG-30 dipolyhydroxystearate, cetyldimethicone copolyol, glycol distearate, glycol dilaurate, diethylene glycoldilaurate, sorbitan trioleate, glycol oleate, glyceryl dilaurate, sorbitan tristearate, propylene glycol stearate, propylene glycol laurate, propylene glycol distearate, sucrose distearate, PEG-3 castor oil, pentaerythrityl monostearate, pentaerythrityl sesquioleate, glyceryl oleate, glyceryl stearate, glyceryl diisostearate, pentaerythrityl monooleate, sorbitan sesquioleate, isostearyl diglyceryl succinate, glyceryl caprate, palm glycerides, cholesterol, lanolin, glyceryl oleate (with 40% monoester), polyglyceryl-2 sesquiisostearate, polyglyceryl-2 sesquioleate, PEG-20 sorbitan beeswax, sorbitan oleate, sorbitan isostearate, trioleyl phosphate, glyceryl stearate and ceteareth-20 (Teginacid from Th. Goldschmidt), sorbitan stearate, PEG-7 hydrogenated castor oil, PEG-5-soyasterol, PEG-6 sorbitan beeswax, glyceryl stearate SE, methylglucose sesquistearates, PEG-10 hydrogenated castor oil, sorbitan palmitate, PEG-22/dodecyl glycol copolymer, polyglyceryl-2 PEG-4 stearate, sorbitan laurate, PEG-4 laurate, polysorbate 61, polysorbate 81, polysorbate 65, polysorbate 80, triceteareth-4 phosphate, triceteareth-4 phosphate and sodium C₁₄₋₁₇ alkyl sec sulfonate (Hostacerin CG from Hoechst), glyceryl stearate and PEG-100 stearates (Arlacel 165 from ICI), polysorbate 85, trilaureth-4 phosphate, PEG-35 castor oil, sucrose stearate, trioleth-8 phosphate, C₁₂₋₁₅ pareth-12, PEG-40 hydrogenated castor oil, PEG-16 soyasterol, polysorbate 80, polysorbate 20, PEG-40 castor oil, sodium cetearyl sulfate, lecithin, laureth-4 phosphate, propylene glycol stearate SE, PEG-25 hydrogenated castor oil, PEG-54 hydrogenated castor oil, glyceryl stearate SE, PEG-6 caprylic/capric glycerides, glyceryl oleate and propylene glycol, glyceryl lanolate, polysorbate 60, glyceryl myristate, glyceryl isostearate and polyglyceryl-3 oleate, glyceryl laurate, PEG-40 sorbitan peroleate, laureth-4, glycerol monostearate, isostearyl glyceryl ether, cetearyl alcohol and sodium cetearyl sulfate, PEG-22 dodecyl glycol copolymer, polyglyceryl-2 PEG-4 stearate, pentaerythrityl isostearate, polyglyceryl-3-diisostearate, sorbitan oleate and hydrogenated castor oil and Cera alba and stearic acid, sodium dihydroxycetyl phosphate and isopropyl hydroxycetyl ether, methylglucose sesquistearate, methylglucose dioleate, sorbitan oleate and PEG-2 hydrogenated castor oil and ozokerite and hydrogenated castor oil, PEG-2 hydrogenated castor oil, PEG-45/dodecyl glycol copolymer, methoxy PEG-22/dodecyl glycol copolymer, hydrogenated cocoglycerides, polyglyceryl-4 isostearate, PEG-40 sorbitan peroleate, PEG-40 sorbitan perisostearate, PEG-8 beeswax, laurylmethicone copolyol, polyglyceryl-2 laurate, stearamidopropyl PG dimonium chloride phosphate, PEG-7 hydrogenated castor oil, triethyl citrate, glyceryl stearate citrate, cetyl phosphate, polyglycerol methylglucose distearate, poloxamer 101, potassium cetyl phosphate, glyceryl isostearate, polyglyceryl-3 diisostearates.

Preferably, for the purposes of the present invention, the further emulsifier(s) is/are chosen from the group of hydrophilic emulsifiers. According to the invention, particular preference is given to mono-, di- and tri-fatty acid esters of sorbitol.

The total amount of further emulsifiers is, according to the invention, advantageously chosen to be less than 5% by weight, based on the total weight of the formulation.

The list of given further emulsifiers which can be used for the purposes of the present invention is not of course intended to be limiting.

Particularly advantageous self-foaming and/or foamable preparations for the purposes of the present invention are free from mono- or diglyceryl fatty acid esters. Particular preference is given to preparations according to the invention which comprise no glyceryl stearate, glyceryl isostearate, glyceryl diisostearate, glyceryl oleate, glyceryl palmitate, glyceryl myristate, glyceryl lanolate and/or glyceryl laurate.

The oil phase of the preparations according to the invention is advantageously chosen from the group of cyclic and/or linear silicone oils and silicone waxes.

Silicone oils are high molecular weight synthetic polymeric compounds in which silicon atoms are joined via oxygen atoms in a chain-like and/or network-like manner and the remaining valances of the silicon are saturated by hydrocarbon radicals (in most cases methyl, more rarely ethyl, propyl, phenyl groups inter alia). Systematically, the silicone oils are referred to as polyorganosiloxanes; the methyl-substituted polyorganosiloxanes, which represent the most significant compounds of this group in terms of amount and are characterized by the following structural formula

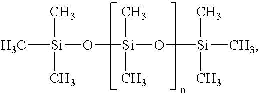

are also referred as polydimethylsiloxane or Dimethicone (INCI). There are dimethicones with different chain lengths or with different molecular weights.

Dimethicones of different chain length and phenyltrimethicones are particularly advantageous linear silicone oils for the purposes of the present invention.

Particularly advantageous polyorganosiloxanes for the purposes of the present invention are also, for example, dimethylpolysiloxane [poly(dimethylsiloxane)], which are obtainable, for example, under the trade names Abil 10 to 10 000 from Th. Goldschmidt. Also advantageous are phenylmethylpolysiloxane (INCI: Phenyl Dimethicone, Phenyl Trimethicone), cyclic silicones (octamethylcyclotetrasiloxane decamethylcyclopentasiloxane), which are referred to according to INCI also as cyclomethicones, amino-modified silicones (INCI: Amodimethicone) and silicone waxes, e.g. polysiloxane-polyalkylene copolymers (INCI: Stearyl Dimethicone and Cetyl Dimethicone) and dialkoxydimethylpolysiloxane (Stearoxy Dimethicone and Behenoxy Stearyl Dimethicone), which are obtainable as various Abil wax grades from Th. Goldschmidt.

Particularly advantageous cyclic silicone oils for the purposes of the present invention are cyclomethicones, in particular cyclomethicone D5 and/or cyclomethicone D6.

The content of the lipid phase is advantageously chosen to be less than 50% by weight, preferably between 1 and 40% by weight, particularly preferably between 5 and 15% by weight, in each case based on the total weight of the foamable preparation. It may in some cases also be advantageous, but is not obligatory, for the lipid phase to comprise up to 40% by weight, based on the total weight of the lipid phase, of further lipids.

Advantageous further lipids for the purposes of the present invention are, for example, all native lipids, such as, for example, olive oil, sunflower oil, soybean oil, groundnut oil, rapeseed oil, almond oil, palm oil, coconut oil, castor oil, wheat germ oil, grape seed oil, thistle oil, evening primrose oil, macadamia nut oil, corn oil, avocado oil and the like, and the lipids having a different polarity listed below:

| | Polarity |
|---|---|
| INCI name | mN/m |
| Cycloparaffin | 49.1 |
| Polydecene | 46.7 |
| Hydrogenated polyisobutene | 44.7 |
| Polydimethylsiloxane | 46.5 |
| Isohexadecane | 43.8 |
| Mineral oil | 43.7 |
| Mineral oil | 43.7 |
| Polydimethylsiloxane | 42.4 |
| Isoeicosane | 41.9 |
| Polydimethylsiloxane | 40.9 |
| Ethoxydiglycol oleate | 40.5 |
| Decyl olivate | 40.3 |
| Dioctylcyclohexane | 39.0 |
| Mineral oil | 38.3 |
| Paraffinum liquidum | 37.6 |
| Isocetyl palmitate | 36.2 |
| Cyclopentasiloxane | 32.3 |
| Octyl isostearate | 31.6 |
| Dicaprylyl carbonate | 31.7 |
| Trimethylhexyl isononanoate | 31.1 |
| 2-Ethylhexyl isononanoate | 31.0 |
| Octyl cocoate | 30.0 |
| Dicaprylyl ether | 30.9 |
| Dihexyl carbonate | 30.9 |
| Polydecene | 30.1 |
| Isodecyl neopentanoate | 29.9 |
| Isohexyl decanoate | 29.7 |
| Isodecyl octanoate | 29.6 |
| Dihexyl ether | 29.2 |
| Isodecyl 3,5,5 trimethyl hexanoate | 29.1 |
| Cetearyl isononanoate | 28.6 |
| Isopropyl palmitate | 28.8 |
| Cyclomethicone | 28.5 |
| Cyclopolydimethylsiloxane | 28.5 |
| Jojoba oil, Buxus chinensis | 26.2 |
| Dimethicone | 26.9 |
| 2-Ethylhexanoic acid 3,5,5 trimethyl ester | 26.2 |
| Open | 25.3 |
| Octyldodecanol | 24.8 |
| Hexyl decanol | 24.3 |
| Isotridecyl 3,5,5 trimethylhexanonanoate | 24.5 |
| Hexyldecanol (+) hexyldecyl laurate | 24.3 |
| Octyl palmitate | 23.1 |
| Octyldodeceyl myristate | 22.1 |
| Macadamia nut oil, Macadamia ternifolia | 22.1 |
| Phenyl trimethicone | 22.7 |
| Butyl octanoic acid | 22.1 |
| Isopropyl stearate | 21.9 |
| C12-15 Alkyl benzoate | 21.8 |
| Butylene glycol Caprylate/caprate | 21.5 |
| Caprylic/capric triglyceride | 21.3 |
| Tricaprylin | 20.2 |
| PEG “Diethylhexanoate/diisononanoate/ethylhexyl | 20.1 |
| isononanoate | |
| Butyl decanol (+) hexyl octanol (+) | 19.8 |
| Hexyl decanol (+) butyl octanol | |
| Tridecyl stearate(+) tridecyl trimellitate(+) dipentaerythrityl | 19.4 |
| hexacaprylate/hexacaprate | |
| Ricinus communis/castor oil | 19.2 |
| Propylene glycol dicaprylate/dicaprate | 18.7 |
| Butyl octanol | 17.4 |
| Stearyl heptanoate | 17.8 |
| Persa gratissima/avocado oil | 14.5 |
| Dibutyl adipate | 14.3 |
| PEG 2 Diethylene hexanoate | 10.1 |
| C12-13 Alkyl lactate | 8.8 |
| Diethylene glycol dioctanoate(/diisononanoate | 8.6 |
| Di-C12/13 Alkyl tartrate | 7.1 |
| Propylene glycol monoisostearate | 6.2 |
| Cocoglycerides | 5.1 |
| Triisostearin | 2.4 |

Of the hydrocarbons, paraffin oil in particular and also further hydrogenated polyolefins, such as hydrogenated polyisobutene, squalane and squalene are to be used advantageously as further lipids for the purposes of the present invention.

The cosmetic and/or dermatological preparations according to the invention and foams obtainable therefrom can have the customary composition. For the purposes of the present invention, skincare preparations are particularly advantageous: they can be used for cosmetic and/or dermatological light protection, and also for the treatment of the skin and/or of the hair and as make-up products in decorative cosmetics. A further advantageous embodiment of the present invention consists in aftersun products.

Corresponding to their structure, cosmetic or topical dermatological compositions can be used, for the purposes of the present invention, for example as skin protection cream, day cream or night cream etc. It may be possible and advantageous to use the compositions according to the invention as a base for pharmaceutical formulations.

Just as emulsions of liquid and solid consistency are used as cosmetic cleansing lotions or cleansing creams, the foams obtainable from the preparations according to the invention can also be “cleansing foams” which can be used, for example, for the removal of make-up or as a mild washing foam, possibly also for bad skin. Such cleansing foams can advantageously also be used as “rinse-off” preparations, which are rinsed from the skin following application.

Foams obtainable from cosmetic and/or dermatological preparations according to the invention can also advantageously be in the form of a foam for care of the hair or of the scalp, in particular a foam for arranging the hair, a foam which is used when blow-drying the hair, a styling foam and treatment foam.

For use, the cosmetic and dermatological preparations according to the invention and/or foams obtainable therefrom are applied to the skin and/or the hair in an adequate amount in the manner customary for cosmetics.

The cosmetic and dermatological preparations according to the invention can comprise cosmetic auxiliaries, as are customarily used in such preparations, e.g. preservatives, preservative assistants, bactericides, perfumes, dyes, pigments which have a coloring action, moisturizers and/or humectants, fillers which improve the feel on the skin, fats, oils, waxes or other customary constituents of a cosmetic or dermatological formulation, such as alcohols, polyols, polymers, foam stabilizers, electrolytes, organic solvents or silicone derivatives.

Advantageous preservatives for the purposes of the present invention are, for example, formaldehyde donors (such as, for example, DMDM hydantoin), iodopropylbutyl carbamates (e.g. those available under the trade names Koncyl-L, Koncyl-S and Konkaben LMB from Lonza), parabens, phenoxyethanol, ethanol, benzoic acid and the like. According to the invention, the preservative system usually also advantageously comprises preservative assistants, such as, for example, octoxyglycerol, glycine soybean etc.

Particularly advantageous preparations are also obtained if antioxidants are used as additives or active ingredients. According to the invention, the preparations advantageously comprise one or more antioxidants. Favorable, but nevertheless optional antioxidants which may be used are all antioxidants customary or suitable for cosmetic and/or dermatological applications.

The antioxidants are advantageously chosen from the group consisting of amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, β-carotene, lycopene) and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate of benzoin resin, rutinic acid and derivatives thereof, ferulic acid and derivatives thereof, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of these listed active ingredients which are suitable according to the invention.

For the purposes of the present invention, water-soluble antioxidants, such as, for example, vitamins, e.g. ascorbic acid and derivatives thereof, can be used particularly advantageously.

A surprising property of the preparations according to the invention is that they are very good vehicles for cosmetic or dermatological active ingredients into the skin, preferred active ingredients being antioxidants which can protect the skin against oxidative stress. Preferred antioxidants here are vitamin E and derivatives thereof, and vitamin A and derivatives thereof.

The amount of antioxidants (one or more compounds) in the preparations is preferably 0.001 to 30% by weight, particularly preferably 0.05 to 20% by weight, in particular 0.1 to 10% by weight, based on the total weight of the preparation.

If vitamin E and/or derivatives thereof are the antioxidant(s), it is advantageous to choose their respective concentrations from the range from 0.001 to 10% by weight, based on the total weight of the formulation.

If vitamin A or vitamin A derivatives, or carotenes or derivatives thereof are the antioxidant(s), it is advantageous to choose their respective concentrations from the range from 0.001 to 10% by weight, based on the total weight of the formulation.

The active ingredients (one or more compounds) can also very advantageously be chosen according to the invention from the group of lipophilic active ingredients, in particular from the following group:

acetylsalicylic acid, atropine, azulene, hydrocortisone and derivatives thereof, e.g. hydrocortisone-17 valerate, vitamins of the B and D series, very favorably vitamin B₁, vitamin B₁₂ and vitamin D₁, but also bisabolol, unsaturated fatty acids, namely the essential fatty acids (often also called vitamin F), in particular gamma-linolenic acid, oleic acid, eicosapentaenoic acid, docosahexaenoic acid and derivatives thereof, chloroamphenicol, caffeine, prostaglandins, thymol, camphor, extracts or other products of a vegetable and animal origin, e.g. evening primrose oil, borage oil or currant seed oil, fish oils, cod-liver oil and also ceramides and ceramide-like compounds, etc.

It is also advantageous to choose the active ingredients from the group of refatting substances, for example purcellin oil, Eucerit® and Neocerit®.

The active ingredient(s) is/are also particularly advantageously chosen from the group of NO synthase inhibitors, particularly if the preparations according to the invention are to be used for the treatment and prophylaxis of the symptoms of intrinsic and/or extrinsic skin aging and for the treatment and prophylaxis of the harmful effects of ultraviolet radiation on the skin.

A preferred NO synthase inhibitor is nitroarginine.

The active ingredient(s) is/are also advantageously chosen from the group which includes catechins and bile esters of catechins and aqueous or organic extracts from plants or parts of plants which have a content of catechins or bile esters of catechins, such as, for example, the leaves of the Theaceae plant family, in particular of the species Camellia sinensis (green tea). Particularly advantageous are typical ingredients thereof (such as e.g. polyphenols or catechins, caffeine, vitamins, sugars, minerals, amino acids, lipids).

Catechins are a group of compounds which are to be regarded as hydrogenated flavones or anthocyanidines and are derivatives of “catechin”(catechol, 3,3′,4′,5,7-flavanpentaol, 2-(3,4-dihydroxyphenyl)chroman-3,5,7-triol). Epicatechin ((2R,3R)-3,3′,4′,5,7-flavanpentaol) is also an advantageous active ingredient for the purposes of the present invention.

Also advantageous are plant extracts with a content of catechins, in particular extracts of green tea, such as e.g. extracts from leaves of plants of the species Camellia spec., very particularly the types of tea Camellia sinenis, C. assamica, C. taliensis and C. irrawadiensis and hybrids of these with, for example, Camellia japonica.

Preferred active ingredients are also polyphenols or catechins from the group (−)-catechin, (+)-catechin, (−)-catechin gallate, (−)-gallocatechin gallate, (+)-epicatechin, (−)-epicatechin, (−)-epicatechin gallate, (−)-epigallocatechin and (−)-epigallocatechin gallate.

Flavone and its derivatives (also often collectively called “flavones”) are also advantageous active ingredients for the purposes of the present invention. They are characterized by the following basic structure (substitution positions are shown):

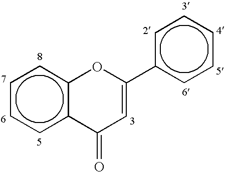

Some of the more important flavones which can also preferably be used in preparations according to the invention are given in the table below:

| | OH substitution positions | | | | | | | |
|---|---|---|---|---|---|---|---|---|
| | 3 | 5 | 7 | 8 | 2′ | 3′ | 4′ | 5′ |
| Flavone | − | − | − | − | − | − | − | − |
| Flavonol | + | − | − | − | − | − | − | − |
| Chrysin | − | + | + | − | − | − | − | − |
| Galangin | + | + | + | − | − | − | − | − |
| Apigenin | − | + | + | − | − | − | + | − |
| Fisetin | + | − | + | − | − | + | + | − |
| Luteolin | − | + | + | − | − | + | + | − |
| Kaempferol | + | + | + | − | − | − | + | − |
| Quercetin | + | + | + | − | − | + | + | − |
| Morin | + | + | + | − | + | − | + | − |
| Robinetin | + | − | + | − | − | + | + | + |
| Gossypetin | + | + | + | + | − | + | + | − |
| Myricetin | + | + | + | − | − | + | + | + |

In nature, flavones are usually in glycosylated form.

According to the invention, the flavonoids are preferably chosen from the group of substances of the generic structural formula

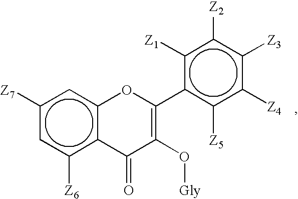

where Z₁ to Z₇, independently of one another, are chosen from the group consisting of H, OH, alkoxy and hydroxyalkoxy, where the alkoxy and hydroxyalkoxy groups can be branched or unbranched and have 1 to 18 carbon atoms, and where Gly is chosen from the group of mono- and oligoglycoside radicals.

According to the invention, the flavonoids can however, also advantageously be chosen from the group of substances of the generic structural formula

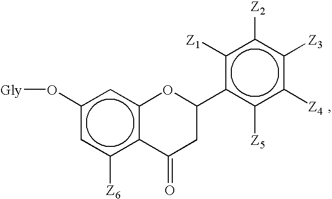

where Z₁ to Z₆, independently of one another, are chosen from the group consisting of H, OH, alkoxy and hydroxyalkoxy, where the alkoxy and hydroxyalkoxy groups can be branched or unbranched and have 1 to 18 carbon atoms, and where Gly is chosen from the group of mono and oligoglycoside radicals.

Preferably, such structures can be chosen from the group of substances of the generic structural formula

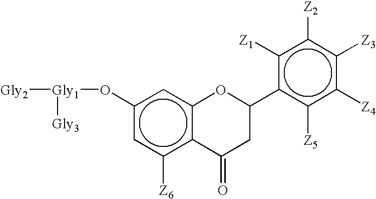

where Gly₁, Gly₂ and Gly₃, independently of one another, are monoglycoside radicals. Gly₂ and Gly₃ can also, individually or together, represent saturations by hydrogen atoms.

Preferably, Gly₁, Gly₂ and Gly₃, independently of one another, are chosen from the group of hexosyl radicals, in particular of rhamnosyl radicals and glucosyl radicals. However, other hexosyl radicals, for example allosyl, altrosyl, galactosyl, gulosyl, idosyl, mannosyl and talosyl, can also be used advantageously in some circumstances. It may also be advantageous according to the invention to use pentosyl radicals.

Z₁ to Z₅ are, independently of one another, advantageously chosen from the group consisting of H, OH, methoxy, ethoxy and 2-hydroxyethoxy, and the flavone glycosides have the structure

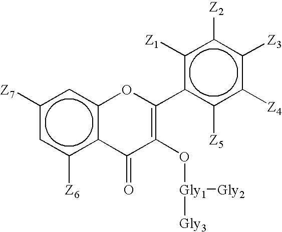

The flavone glycosides according to the invention are particularly advantageously chosen from the group given by the following structure:

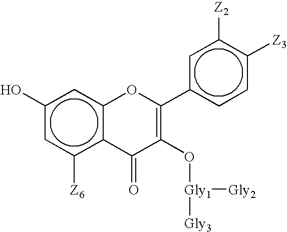

where Gly₁, Gly₂ and Gly₃, independently of one another, are monoglycoside radicals. Gly₂ and Gly₃ can also, individually or together, represent saturations by hydrogen atoms.

Preferably, Gly₁, Gly₂ and Gly₃, independently of one another, are chosen from the group of hexosyl radicals, in particular of rhamnosyl radicals and glucosyl radicals. However, other hexosyl radicals, for example allosyl, altrosyl, galactosyl, gulosyl, idosyl, mannosyl and talosyl, can also advantageously be used in some circumstances. It may also be advantageous according to the invention to use pentosyl radicals.

For the purposes of the present invention, it is particularly advantageous to choose the flavone glucoside(s) from the group consisting of α-glucosylrutin, α-glucosylmyricetin, α-glucosylisoquercitrin, α-glucosylisoquercetin and α-glucosylquercitrin.

Particular preference is given, according to the invention, to α-glucosylrutin.

Also advantageous according to the invention are naringin (aurantin, naringenin-7-rhamnoglucoside), hesperidin (3′5,7-trihydroxy-4′-methoxyflavanone-7-rutinoside, hesperidoside, hesperetin-7-O-rutinoside), rutin (3,3′4′,5,7-pentahydroxyflyvone-3-rutinoside, quercetin-3-rutinoside, sophorin, birutan, rutabion, taurutin, phytomelin, melin), troxerutin (3,5-dihydroxy-3′4′,7-tris(2-hydroxyethoxy)flavone-3-(6-O-(6 -deoxy -αL-mannopyranosyl )-β-D-glucopyranoside)), monoxerutin (3,3′,4′,5-tetrahydroxy-7-(2-hydroxyethoxy) flavone-3-(6-O-(6-deoxy-α-L-mannopyranosyl )-β-D-glucopyranoside)), dihydrorobinetin (3,3′,4′,5′,7-pentahydroxyflavanone), taxifolin (3,3′,4′,5,7-pentahydroxy-flavanone), eriodictyol-7-glucoside (3′,4′,5,7-tetrahydroxyflavanone-7 glucoside), flavanomarein (3′,4′,7,8-tetrahydroxyflavanone-7 glucoside) and isoquercetin (3,3′,4!,5,7-pentahydroxyflavanone-3-(β-D-glucopyranoside).

It is also advantageous to choose the active ingredient(s) from the group of ubiquinones and plastoquinones.

Ubiquinones are distinguished by the structural formula

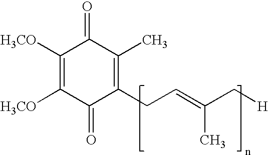

and are the most widespread and thus the most investigated bioquinones. Ubiquinones are referred to depending on the number of isoprene units linked in the side chain as Q-1, Q-2, Q-3 etc., or depending on the number of carbon atoms, as U-5, U-10, U-15 etc. They preferably appear with certain chain lengths, e.g. in some microorganisms and yeasts where n=6. In most mammals including man, Q10 predominates.

Coenzyme Q10 is particularly advantageous and is characterized by the following structural formula:

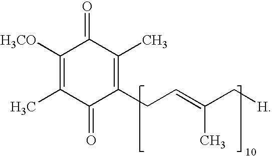

Plastoquinones have the general structural formula

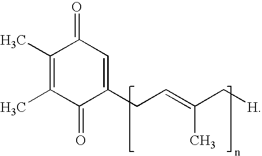

Plastoquinones differ in the number n of isoprene radicals and are referred to accordingly, e.g. PQ-9 (n=9). In addition, other plastoquinones with varying substituents on the quinone ring exist.

Creatine and/or creatine derivatives are preferred active ingredients for the purposes of the present invention. Creatine is characterized by the following structure:

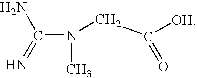

Preferred derivatives are creatine phosphate and creatine sulfate, creatine acetate, creatine ascorbate and the derivatives esterified at the carboxyl group with mono- or polyfunctional alcohols.

A further advantageous active ingredient is L-carnitine [3-hydroxy-4-(trimethylammonio) butyrobetaine]. Acylcarnitines which chosen from the group of substances of the following general structural formula

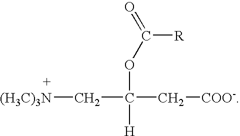

where R is chosen from the group of branched and unbranched alkyl radicals having up to 10 carbon atoms, are advantageous active ingredients for the purposes of the present invention. Preference is given to propionylcarnitine and, in particular, acetylcarnitine. Both enantiomers (D and L form) are to be used advantageously for the purposes of the present invention. It may also be advantageous to use any enantiomer mixtures, for example a racemate of D and L form.

Further advantageous active ingredients are sericoside, pyridoxol, vitamin K, biotin and aroma substances.

The list of said active ingredients and active ingredient combinations which can be used in the preparations according to the invention is, of course, not intended to be limiting. The active ingredients can be used individually or in any combinations with one another.

Skin aging is caused e.g. by endogenous, genetically determined factors. As a result of aging, the epidermis and dermis experience e.g. the following structural damage and functional disorders, which can also be covered by the term “senile xerosis”:

a) dryness, roughness and formation of (dryness) wrinkles,

b) itching and

c) reduced refatting by sebaceous glands (e.g. after washing).

Exogenous factors, such as UV light and chemical noxae, can have a cumulative effect and, for example, accelerate or add to the endogenous aging processes. The epidermis and dermis experience, in particular as a result of exogenous factors, e.g. the following structural damage and functional disorders in the skin, which go beyond the degree and quality of the damage in the case of chronological aging:

d) visible vascular dilations (telangiectases, cuperosis);

e) flaccidity and formation of wrinkles;

f) local hyperpigmentation, hypopigmentation and abnormal pigmentation (e.g. age spots) and

g) increased susceptibility to mechanical stress (e.g. cracking).

Surprisingly, selected formulations according to the invention can also have an anti-wrinkle action or considerably increase the action of known anti-wrinkle active ingredients. Accordingly, for the purposes of the invention, formulations are particularly advantageously suitable for the prophylaxis and treatment of cosmetic or dermatological skin changes, as arise, for example, during skin aging. They are also advantageously suitable for combating the development of dry or rough skin.

In a particular embodiment, the present invention thus relates to products for the care of skin aged in a natural manner, and for the treatment of the secondary damage of light aging, in particular the phenomena listed under a) to g).

The water phase of the preparations according to the invention can advantageously comprise customary cosmetic auxiliaries, such as, for example, alcohols, in particular those of low carbon number, preferably ethanol and/or isopropanol, diols or polyols of low carbon number, and ethers thereof, preferably propylene glycol, glycerol, ethylene glycol, ethylene glycol monoethyl or monobutyl ether, propylene glycol monomethyl, monoethyl or monobutyl ether, diethyleneglycol monomethyl or monoethyl ether and analogous products, polymers, foam stabilizers, electrolytes and moisturizers.

Moisturizers is the term used to describe substances or mixtures of substances which, following application or distribution on the surface of the skin, confer on cosmetic or dermatological preparations the property of reducing the moisture loss by the horny layer (also called transepidermal water loss (TEWL)) and/or have a beneficial effect on the hydration of the horny layer.

Advantageous moisturizers for the purposes of the present invention are, for example, glycerol, lactic acid, pyrrolidonecarboxylic acid and urea. In addition, it is particularly advantageous to use polymeric moisturizers from the group of polysaccharides which are soluble in water and/or swellable in water and/or gellable using water. Particularly advantageous are, for example, hyaluronic acid, chitosan and/or a fucose-rich polysaccharide which is listed in Chemical Abstracts under the registry number 178463-23-5 and is available, for example, under the name Fucogel®1000 from SOLABIA S.A.

The cosmetic and dermatological preparations according to the invention can comprise dyes and/or color pigments, particularly when they are in the form of decorative cosmetics. The dyes and color pigments can be chosen from the corresponding positive list of the Cosmetics Directive or the EC list of cosmetic colorants. In most cases they are identical to the dyes approved for foods. Advantageous color pigments are, for example, titanium dioxide, mica, iron oxides (e.g. Fe₂O₃, Fe₃O₄, FeO(OH)) and/or tin oxide. Advantageous dyes are, for example, carmine, Berlin blue, chrome oxide green, ultramarine blue and/or manganese violet. It is particularly advantageous to choose the dyes and/or color pigments from the following list. The Colour Index Numbers (CIN) are taken from the Rowe Colour Index, 15 3rd Edition, Society of Dyers and Colourists, Bradford, England, 1971.

| Chemical or other name | CIN | Color |
|---|---|---|
| Pigment Green | 10006 | green |
| Acid Green 1 | 10020 | green |
| 2,4-Dinitrohydroxynaphthalene-7-sulfonic acid | 10316 | yellow |
| Pigment Yellow 1 | 11680 | yellow |
| Pigment Yellow 3 | 11710 | yellow |
| Pigment Orange 1 | 11725 | orange |
| 2,4-Dihydroxyazobenzene | 11920 | orange |
| Solvent Red 3 | 12010 | red |
| 1-(2′-Chloro-4′-nitro-1′-phenylazo)-2- | 12085 | red |
| hydroxynaphthalene | | |
| Pigment Red 3 | 12120 | red |
| Ceres red; Sudan red; Fat Red G | 12150 | red |
| Pigment Red 112 | 12370 | red |
| Pigment Red 7 | 12420 | red |
| Pigment Brown 1 | 12480 | brown |
| 4-(2′-Methoxy-5′-sulfodiethylamido-1′-phenylazo)- | 12490 | red |
| 3-hydroxy-5″-chloro-2″,4″-dimethoxy- | | |
| 2-naphthanilide | | |
| Disperse Yellow 16 | 12700 | yellow |
| 1-(4-Sulfo-1-phenylazo)-4-aminobenzene-5-sulfonic | 13015 | yellow |
| acid | | |
| 2,4-Dihydroxyazobenzene-4′-sulfonic acid | 14270 | orange |
| 2-(2,4-Dimethylphenylazo-5-sulfo)-1- | 14700 | red |
| hydroxynaphthalene-4-sulfonic acid | | |
| 2-(4-Sulfo-1-naphthylazo)-1-naphthol-4-sulfonic acid | 14720 | red |
| 2-(6-Sulfo-2,4-xylylazo)-1-naphthol-5-sulfonic acid | 14815 | red |
| 1-(4′-Sulfophenylazo)-2-hydroxynaphthalene | 15510 | orange |
| 1-(2-Sulfo-4-chloro-5-carboxy-1-phenylazo)-2- | 15525 | red |
| hydroxynaphthalene | | |
| 1-(3-Methylphenylazo-4-sulfo)-2-hydroxynaphthalene | 15580 | red |
| 1-(4′,(8′)-Sulfonaphthylazo)-2-hydroxynaphthalene | 15620 | red |
| 2-Hydroxy-1,2′-azonaphthalene-1′-sulfonic acid | 15630 | red |
| 3-Hydroxy-4-phenylazo-2-naphthylcarboxylic acid | 15800 | red |
| 1-(2-Sulfo-4-methyl-1-phenylazo)-2- | 15850 | red |
| naphthylcarboxylic acid | | |
| 1-(2-Sulfo-4-methyl-5-chloro-1-phenylazo)-2- | 15865 | red |
| hydroxynaphthalene-3-carboxylic acid | | |
| 1-(2-Sulfo-1-naphthylazo)-2-hydroxynaphthalene-3- | 15880 | red |
| carboxylic acid | | |
| 1-(3-Sulfo-1-phenylazo)-2-naphthol-6-sulfonic acid | 15980 | orange |
| 1-(4-Sulfo-1-phenylazo)-2-naphthol-6-sulfonic acid | 15985 | yellow |
| Allura Red | 16035 | red |
| 1-(4-Sulfo-1-naphthylazo)-2-naphthol-3,6-disulfonic | 16185 | red |
| acid Acid Orange 10 | 16230 | orange |
| 1-(4-Sulfo-1-naphthylazo)-2-naphthol-6,8-disulfonic | 16255 | red |
| acid | | |
| 1-(4-Sulfo-1-naphthylazo)-2-naphthol-3,6,8- | 16290 | red |
| trisulfonic acid | | |
| 8-Amino-2-phenylazo-1-naphthol-3,6-disulfonic acid | 17200 | red |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-Sulfo-1-phenylazo)-1-(4-sulfophenyl)-5-hydroxy- | 19140 | yellow |
| pyrazolone-3-carboxylic acid | | |
| Pigment Yellow 16 | 20040 | yellow |
| 2,6-(4′-Sulfo-2″, 4″-dimethyl)bisphenylazo)- | 20170 | orange |
| 1,3-dihydroxy-benzene | | |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-Sulfo-1″-phenylazo)-7′-sulfo-1′-naphthylazo]- | 27755 | black |
| 1-hydroxy-7-aminonaphthalene-3,6-disulfonic acid | | |
| 4′-[(4″-Sulfo-1″-phenylazo)-7′-sulfo-1′- | 28440 | black |
| naphthylazo]-1-hydroxy-8-acetylaminonaphthalene- | | |
| 3,5-disulfonic acid | | |
| Direct Orange 34, 39, 44, 46, 60 | 40215 | orange |
| Food Yellow | 40800 | orange |
| trans-β-Apo-8′-carotenaldehyde (C₃₀) | 40820 | orange |
| trans-Apo-8′-carotenic acid (C₃₀)-ethyl ester | 40825 | orange |
| Canthaxanthin | 40850 | orange |
| Acid Blue 1 | 42045 | blue |
| 2,4-Disulfo-5-hydroxy-4′-4″- | 42051 | blue |
| bis(diethylamino)triphenylcarbinol | | |
| 4-[(4-N-Ethyl-p-sulfobenzylamino)phenyl(4-hydroxy- | 42053 | green |
| 2-sulfophenyl)(methylene)-1-(N-ethyl-N-p- | | |
| sulfobenzyl)-2,5-cyclohexadienimine] | | |
| Acid Blue 7 | 42080 | blue |
| (N-Ethyl-p-sulfobenzylamino)phenyl(2- | 42090 | blue |
| sulfophenyl)methylene-(N-ethyl-N-p- | | |
| sulfobenzyl)Δ^{2,5}-cyclohexadienimine | | |
| Acid Green 9 | 42100 | green |
| Diethyldisulfobenzyl-di-4-amino-2-chloro-di-2-methyl- | 42170 | green |
| fuchsonimmonium | | |
| Basic Violet 14 | 42510 | violet |
| Basic Violet 2 | 42520 | violet |
| 2′-Methyl-4′-(N-ethyl-N-m-sulfobenzyl)amino-4″-(N- | 42735 | blue |
| diethyl)amino-2-methyl-N-ethyl-N-m- | | |
| sulfobenzylfuchsonimmonium | | |
| 4′-(N-Dimethyl)amino-4″-(N-phenyl)aminonaphtho- | 44045 | blue |
| N-dimethyl-fuchsonimmonium | | |
| 2-Hydroxy-3,6-disulfo-4,4′-bisdimethylaminonaphtho- | 44090 | green |
| fuchsonimmonium | | |
| Acid Red 52 | 45100 | red |
| 3-(2′-Methylphenylamino)-6-(2′-methyl-4′- | 45190 | violet |
| sulfophenylamino)-9-(2″- | | |
| carboxyphenyl)xanthenium salt | | |
| Acid Red 50 | 45220 | red |
| Phenyl-2-oxyfluorone-2-carboxylic acid | 45350 | yellow |
| 4,5-Dibromofluorescein | 45370 | orange |
| 2,4,5,7-Tetrabromofluorescein | 45380 | red |
| Solvent Dye | 45396 | orange |
| Acid Red 98 | 45405 | red |
| 3′,4′,5′,6′-Tetrachloro-2,4,5,7-tetrabromofluorescein | 45410 | red |
| 4,5-Diiodofluorescein | 45425 | red |
| 2,4,5,7-Tetraiodofluorescein | 45430 | red |
| Quinophthalone | 47000 | yellow |
| Quinophthalonedisulfonic acid | 47005 | yellow |
| Acid Violet 50 | 50325 | violet |
| Acid Black 2 | 50420 | black |
| Pigment Violet 23 | 51319 | violet |
| 1,2-Dioxyanthraquinone, calcium-aluminum complex | 58000 | red |
| 3-Oxypyrene-5,8,10-sulfonic acid | 59040 | green |
| 1-Hydroxy-4-N-phenylaminoanthraquinone | 60724 | violet |
| 1-Hydroxy-4-(4′-methylphenylamino)anthraquinone | 60725 | violet |
| Acid Violet 23 | 60730 | violet |
| 1,4-Di(4′-methylphenylamino)anthraquinone | 61565 | green |
| 1,4-Bis(o-sulfo-p-toluidino)anthraquinone | 61570 | green |
| Acid Blue 80 | 61585 | blue |
| Acid Blue 62 | 62045 | blue |
| N,N′-Dihydro-1,2,1′,2′-anthraquinone azine | 69800 | blue |
| Vat Blue 6; Pigment Blue 64 | 69825 | blue |
| Vat Orange 7 | 71105 | orange |
| Indigo | 73000 | blue |
| Indigo-disulfonic acid | 73015 | blue |
| 4,4′-Dimethyl-6,6′-dichlorothioindigo | 73360 | red |
| 5,5′-Dichloro-7,7′-dimethylthioindigo | 73385 | violet |
| Quinacridone Violet 19 | 73900 | violet |
| Pigment Red 122 | 73915 | red |
| Pigment Blue 16 | 74100 | blue |
| Phthalocyanine | 74160 | blue |
| Direct Blue 86 | 74180 | blue |
| Chlorinated phthalocyanine | 74260 | green |
| Natural Yellow 6,19; Natural Red 1 | 75100 | yellow |
| Bixin, Norbixin | 75120 | orange |
| Lycopene | 75125 | yellow |
| trans-alpha-, beta- and gamma-carotene | 75130 | orange |
| Keto- and/or hydroxyl derivates of carotene | 75135 | yellow |
| Guanine or pearlescent agent | 75170 | white |
| 1,7-Bis(4-hydroxy-3-methoxyphenyl)-1,6- | 75300 | yellow |
| heptadiene-3,5-dione | | |
| Complex salt (Na, Al, Ca) of carminic acid | 75470 | red |
| Chlorophyll a and b; copper compounds of | 75810 | green |
| chlorophylls and chlorophyllins | | |
| Aluminum | 77000 | white |
| Hydrated alumina | 77002 | white |
| Hydrous aluminum silicates | 77004 | white |
| Ultramarine | 77007 | blue |
| Pigment Red 101 and 102 | 77015 | red |
| Barium sulfate | 77120 | white |
| Bismuth oxychloride and its mixtures with mica | 77163 | white |
| Calcium carbonate | 77220 | white |
| Calcium sulfate | 77231 | white |
| Carbon | 77266 | black |
| Pigment black 9 | 77267 | black |
| Carbo medicinalis vegetabilis | 77268:1 | black |
| Chromium oxide | 77288 | green |
| Chromium oxide, hydrous | 77289 | green |
| Pigment Blue 28, Pigment Green 14 | 77346 | green |
| Pigment Metal 2 | 77400 | brown |
| Gold | 77480 | brown |
| Iron oxides and hydroxides | 77489 | orange |
| Iron oxide | 77491 | red |
| Hydrated iron oxide | 77492 | yellow |
| Iron oxide | 77499 | black |
| Mixtures of iron (II) and iron(III)hexacyanoferrate | 77510 | blue |
| Pigment White 18 | 77713 | white |
| Manganese animonium diphosphate | 77742 | violet |
| Manganese phosphate; Mn₃(PO₄)₂.7 H20 | 77745 | red |
| Silver | 77820 | white |
| Titanium dioxide and its mixtures with mica | 77891 | white |
| Zinc oxide | 77947 | white |
| 6,7-Dimethyl-9-(1′-D-ribityl)isoalloxazine, lactoflavine | | yellow |
| Sugar coloring | | brown |
| Capsanthin, capsorubin | | orange |
| Betanin | | red |
| Benzopyrylium salts, anthocyans | | red |
| Aluminum, zinc, magnesium and calcium stearate | | white |
| Bromothymol blue | | blue |
| Bromocresol green | | green |
| Acid Red 195 | | red |

If the formulations according to the invention are in the form of products, which are intended for use in the facial area, it is favorable to choose one or more substances from the following group as the dye: 2,4-dihydroxyazobenzene, 1-(2′-chloro-4′-nitro-1′-phenylazo) -2phenylazo)-2-hydroxynaphthalene, Ceres Red, 2-(4-sulfo-1-naphthylazo)-1-naphthol-4-sulfonic acid, calcium salt of 2-hydroxy-1,2′-azonaphthalene-1′-sulfonic acid, calcium and barium salts of 1-(2-sulfo-4-methyl-1-phenylazo)-2-naphthylcarboxylic acid, calcium salt of 1-(2-sulfo-1-naphthylazo)-2-hydroxynaphthalene-3-carboxylic acid, aluminum salt of 1-(4-sulfo-1-phenylazo)-2-naphthol-6-sulfonic acid, aluminum salt of 1-(4-sulfo-1-naphthylazo)-2-naphthol-3,6-disulfonic acid, 1-(4-sulfo-1-naphthylazo)-2-naphthol-6,8-disulfonic acid, aluminum salt of 4-(4-sulfo-1-phenylazo)-1-(4-sulfophenyl)-5-hydroxy-pyrazolone-3-carboxylic acid, aluminum and zirconium salts of 4,5-dibromofluorescein, aluminum and zirconium salts of 2,4,5,7-tetrabromofluorescein, 3′,4′,5′,6′-tetrachloro-2,4,5,7-tetrabromofluorescein and its aluminum salt, aluminum salt of 2,4,5,7-tetraiodofluorescein, aluminum salt of quinophthalone disulfonic acid, aluminum salt of indigo disulfonic acid, red and black iron oxide (CIN: 77 491 (red) and 77 499 (black)), iron oxide hydrate (CIN: 77 492), manganese ammonium diphosphate and titanium dioxide.

Also advantageous are oil-soluble natural dyes, such as, for example, paprika extracts, β-carotene or cochenille.

Also advantageous for the purposes of the present invention are formulations with a content of pearlescent pigments. Preference is given in particular to the types of pearlescent pigments listed below:

1. Natural pearlescent pigments, such as, for example

“pearl essence” (guanine/hypoxanthin mixed crystals from fish scales) and

“mother of pearl” (ground mussel shells)

2. Monocrystalline pearlescent pigments, such as, for example, bismuth oxychloride (BiOCl)

3. Layer-substrate pigments: e.g. mica/metal oxide

Bases for pearlescent pigments are, for example, pulverulent pigments or castor oil dispersions of bismuth oxychloride and/or titanium dioxide, and bismuth oxychloride and/or titanium dioxide on mica. The luster pigment listed under CIN 77163, for example, is particularly advantageous.

Also advantageous are, for example, the following types of pearlescent pigment based on mica/metal oxide:

| Group | Coating/layer thickness | Color |
|---|---|---|
| Silver-white pearlescent | TiO₂: 40-60 nm | silver |
| pigments | | |
| Interference pigments | TiO₂: 60-80 nm | yellow |
| | TiO₂: 80-100 nm | red |
| | TiO₂: 100-140 nm | blue |
| | TiO₂: 120-160 nm | green |
| Color luster pigments | Fe₂O₃ | bronze |
| | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | gold shades |
| | TiO₂/Cr₂O₃ | green |
| | TiO₂/Berlin blue | deep blue |
| | TiO₂/carmine | red |

Particular preference is given, for example, to the pearlescent pigments obtainable from Merck under the trade names Timiron, Colorona or Dichrona.

The list of given pearlescent pigments is not of course intended to be limiting. Pearlescent pigments which are advantageous for the purposes of the present invention are obtainable by numerous methods known per se. For example, other substrates apart from mica can be coated with further metal oxides, such as, for example, silica and the like. SiO₂ particles coated with, for example, TiO₂ and Fe₂O₃ (“ronaspheres”), which are marketed by Merck and are particularly suitable for the optical reduction of fine lines are advantageous.

It can moreover be advantageous to dispense completely with a substrate such as mica. Particular preference is given to iron pearlescent pigments prepared without the use of mica. Such pigments are obtainable, for example, under the trade name Sicopearl Kupfer 1000 from BASF.

In addition, also particularly advantageous are effect pigments which are obtainable under the trade name Metasome Standard/Glitter in various colors (yellow, red, green, blue) from Flora Tech. The glitter particles are present here in mixtures with various auxiliaries and dyes (such as, for example, the dyes with the Colour Index (Cl) Numbers 19140, 77007, 77289, 77491).

The dyes and pigments may be present either individually or in a mixture, and can be mutually coated with one another, different coating thicknesses generally giving rise to different color effects. The total amount of dyes and color-imparting pigments is advantageously chosen from the range from e.g. 0.1% by weight to 30% by weight, preferably from 0.5 to 15% by weight, in particular from 1.0 to 10% by weight, in each case based on the total weight of the preparations.

For the purposes of the present invention, it is also advantageous to provide cosmetic and dermatological preparations whose main purpose is not protection against sunlight, but which nevertheless have a content of UV protection substances. Thus, for example, UV-A and/or UV-B filter substances are usually incorporated into day creams or make-up products. UV protection substances, like antioxidants, and, if desired, preservatives, also constitute effective protection of the preparations themselves against spoilage. Also favorable are cosmetic and dermatological preparations in the form of a sunscreen.

Accordingly, for the purposes of the present invention, as well as comprising one or more UV filter substances according to the invention, the preparations additionally comprise at least one further UV-A and/or UV-B filter substance. The formulations may, although not necessarily, optionally also comprise one or more organic and/or inorganic pigments as UV filter substances which may be present in the water and/or oil phase.

Preferred inorganic pigments are metal oxides and/or other metal compounds which are insoluble or virtually insoluble in water, in particular oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals and mixtures of such oxides. Barium sulfate (BaSO₄) is also advantageous for the purposes of the present invention.

For the purposes of the present invention, such pigments may advantageously be surface-treated (“coated”), the intention being to form or retain, for example, an amphiphilic or hydrophobic character. This surface treatment can consist in providing the pigments with a thin hydrophobic layer by processes known per se.

Advantageous according to the invention are, e.g., titanium dioxide pigments which have been coated with octylsilanol. Suitable titanium dioxide particles are available under the trade name T805 from Degussa. Also particularly advantageous are TiO₂ pigments coated with aluminum stearate, e.g. those available under the trade name MT 100 T from TAYCA.

A further advantageous coating of the inorganic pigments consists of dimethyl-polysiloxane (also: dimethicone), a mixture of completely methylated, linear siloxane polymers which have been terminally blocked with trimethylsiloxy units. Particularly advantageous for the purposes of the present invention are zinc oxide pigments which have been coated in this way.

Also advantageous is a coating of the inorganic pigments with a mixture of dimethylpolysiloxane, in particular dimethylpolysiloxane having an average chain length of from 200 to 350 dimethylsiloxane units, and silica gel, which is also referred to as simethicone. In particular, it is advantageous for the inorganic pigments to be additionally coated with aluminum hydroxide or aluminum oxide hydrate (also: alumina, CAS No.: 1333-84-2). Particularly advantageous are titanium dioxides which have been coated with simethicone and alumina, it also being possible for the coating to comprise water. An example thereof is the titanium dioxide available under the trade name Eusolex T2000 from Merck.

An advantageous organic pigment for the purposes of the present invention is 2,2′-methylenebis(6-(2H-benzotriazol-2-yl)-4-(1,1,3,3-tetramethylbutyl)phenol) [INCI: bisoctyltriazole], which is characterized by the chemical structural formula

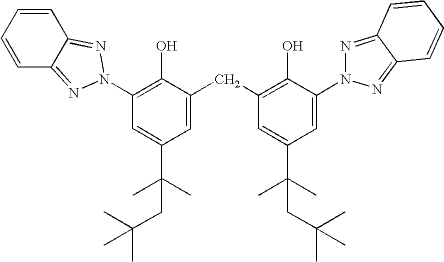

and is available under the trade name Tinosorb® M from CIBA-Chemikalien GmbH.

Preparations according to the invention advantageously comprise substances which absorb UV radiation in the UV-A and/or UV-B range, the total amount of filter substances being, for example, from 0.1% by weight to 30% by weight, preferably from 0.5 to 20% by weight, in particular from 1.0 to 15.0% by weight, based on the total weight of the preparations, in order to provide cosmetic preparations which protect the hair and the skin from the entire range of ultraviolet radiation. They can also be used as sunscreens for the hair or the skin.

Advantageous UV-A filter substances for the purposes of the present invention are dibenzoylmethane derivatives, in particular 4-(tert-butyl)-4′-methoxydibenzoylmethane (CAS No. 70356-09-1), which is sold by Givaudan under the name Parsol®1789 and by Merck under the trade name Eusolex®9020.

Further advantageous UV-A filter substances are phenylene-1,4-bis(2-benzimidazyl)-3,3′-5,5′-tetrasulfonic acid:

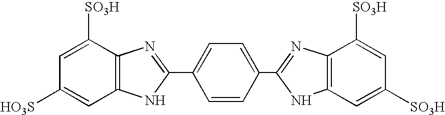

and its salts, particularly the corresponding sodium, potassium or triethanolammonium salts, in particular phenylene-1,4-bis(2-benzimidazyl)-3,3′-5,5′-tetrasulfonic bis-sodium salt:

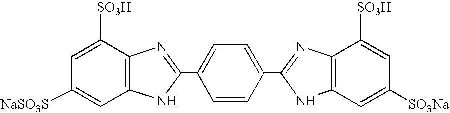

with the INCI name Bisimidazylate, which is available, for example, under the trade name Neo Heliopan AP from Haarmann & Reimer.

Also advantageous are 1,4-di(2-oxo-10-sulfo-3-bornylidenemethyl)benzene and salts thereof (in particular the corresponding 10-sulfato compounds, in particular the corresponding sodium, potassium or triethanolammonium salt), which is also referred to as benzene-1,4-di(2-oxo-3-bornylidenemethyl-10-sulfonic acid) and is characterized by the following structure:

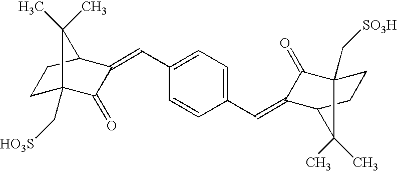

Advantageous UV filter substances for the purposes of the present invention are also broadband filters, i.e. filter substances which absorb both UV-A and also UV-B radiation.

Advantageous broadband filters or UV-B filter substances are, for example, bisresorcinyltriazine derivatives having the following structure:

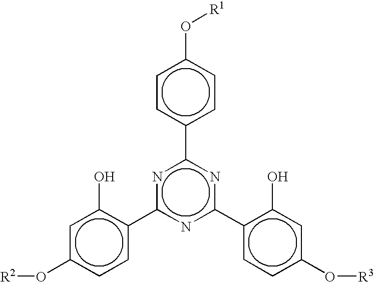

where R¹, R² and R³ independently of one another are chosen from the group of branched and unbranched alkyl groups having 1 to 10 carbon atoms, or are a single hydrogen atom. Particular preference is given to 2,4-bis{[4-(2-ethylhexyloxy)-2-hydroxy]phenyl}-6-(4-methoxyphenyl)-1,3,5-triazine (INCI: Aniso Triazine), which is available under the trade name Tinosorb® S from CIBA-Chemikalien GmbH

For the purposes of the present invention, particularly advantageous preparations which are characterized by high or very high UV-A protection preferably comprise two or more UV-A and/or broadband filters, in particular dibenzoylmethane derivatives [for example 4-(tert-butyl)-4′-methoxydibenzoylmethane], benzotriazole derivatives [for example 2,2′-methylenebis(6-(2H-benzotriazol-2-yl)-4-(1,1,3,3-tetramethylbutyl)phenol)], phenylene-1,4-bis(2-benzimidazyl)-3,3′-5,5′-tetrasulfonic acid and/or its salts, 1 ,4-di(2-oxo-1 0-sulfo-3-bornylidenemethyl)benzene and/or salts thereof and/or 2,4-bis{[4-(2-ethylhexyloxy)-2-hydroxy]phenyl}-6-(4-methoxyphenyl)-1,3,5-triazine, in each case individually or in any combinations with one another.

Other UV filter substances, which have the structural formula

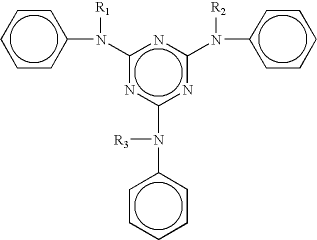

are also advantageous UV filter substances for the purposes of the present invention, for example the s-triazine derivatives described in European laid-open specification EP 570 838 A1, whose chemical structure is expressed by the generic formula

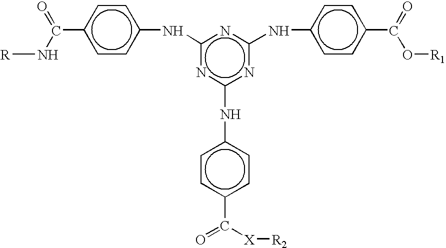

where

R is a branched or unbranched C₁-C₁₈-alkyl radical, a C₅-C₁₂-cycloalkyl radical, optionally substituted with one or more C₁-C₄-alkyl groups,

X is an oxygen atom or an NH group,

R₁ is a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula

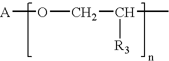

in which

A is a branched or unbranched C₁-C₁₈-alkyl radical, a C₅-C₁₂-cycloalkyl or aryl radical, optionally substituted by one or more C₁-C₄-alkyl groups,

R₃ is a hydrogen atom or a methyl group, n is a number from 1 to 10,

R₂ is a branched or unbranched C₁-C₁₈-alkyl radical, a C₅-C₁₂-cycloalkyl radical, optionally substituted by one or more C₁-C₄-alkyl groups, when X is the NH group, and a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula

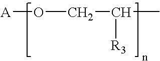

in which

A is a branched or unbranched C₁-C₁₈-alkyl radical, a C₅-C₁₂-cycloalkyl or aryl radical, optionally substituted by one or more C₁-C₄-alkyl groups,

R₃ is a hydrogen atom or a methyl group,

n is a number from 1 to 10,

when X is an oxygen atom.

A particularly preferred UV filter substance for the purposes of the present invention is also an unsymmetrically substituted s-triazine, the chemical structure of which is expressed by the formula

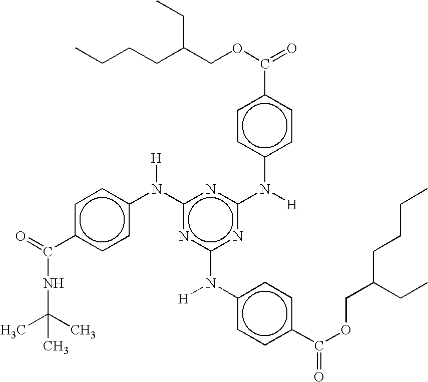

and which is also referred to below as dioctylbutylamidotriazone (INCI: Dioctylbut-amidotriazone), and is available under the trade name UVASORB HEB from Sigma 3V.

Also advantageous for the purposes of the present invention is a symmetrically substituted s-triazine, tris(2-ethylhexyl) 4,4′,4″-(1,3,5-triazine-2,4,6-triyltriimino)tris-benzoate, synonym: 2,4,6-tris[anilino-(p-carbo-2′-ethyl-1′-hexyloxy)]-1,3,5-triazine (INCI: Octyl Triazone), which is marketed by BASF Aktiengesellschaft under the trade name UVINUL® T 150.

European laid-open specification 775 698 also describes preferred bisresorcinyltriazine derivatives, the chemical structure of which is expressed by the generic formula

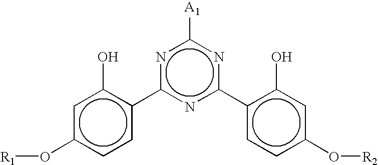

where R₁, R₂ and A₁ represent very different organic radicals.

Also advantageous for the purposes of the present invention are 2,4-bis{[4-(3-sulfonato)-2-hydroxypropyloxy)-2-hydroxy]phenyl}-6-(4-methoxyphenyl)- 1,3 ,5-triazine sodium salt, 2 ,4-bis{[4-(3-(2-propyloxy)-2-hydroxypropyloxy)-2-hyd roxy]phenyl}-6-(4methoxyphenyl)-1,3,5-triazine, 2,4-bis{[4-(2-ethylhexyloxy)-2-hydroxy]phenyl}-6-[4-(2-methoxyethylcarboxyl)phenylamino]-1,3,5-triazine, 2,4-bis{[4-(3-(2-propyloxy)-2 -hydroxypropyloxy)-2-hydroxy]phenyl}-6-[4-(2-ethylcarboxyl)phenylamino]-1,3,5-triazine, 2,4-bis{[4-(2-ethylhexyloxy)-2-hyd roxy] phenyl}-6-( 1-methylpyrrol-2-yl)-1 ,3,5-b bis{[4-tris(trimethylsiloxysilylpropyloxy)-2-hydroxy]phenyl}-6-(4-methoxyphenyl)-1,3,5 -triazine, 2 ,4-bis{[4-(2″-methyl propenyloxy)-2-hyd roxy] phenyl}-6-(4-methoxyphenyl ) -1,3,5-triazine and 2,4-bis{[4-(1′,1′,1′,3′,5′,5′,5′-heptamethylsiloxy-2″-methylpropyloxy)-2-hydroxy] phenyl}-6-(4-methoxyphenyl )-1,3, 5-triazine.

An advantageous broadband filter for the purposes of the present invention is 2,2′-methylenebis(6-(2H-benzotriazol-2-yl)-4-( 1,1,3 ,3-tetramethyl butyl )phenol), which is characterized by the chemical structural formula

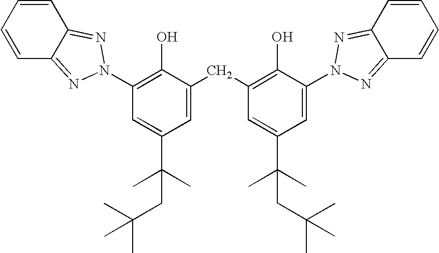

and is available under the trade name Tinosorb® M from CIBA-Chemikalien GmbH.

Another advantageous broadband filter for the purposes of the present invention is 2-(2H-benzotriazol-2-yl)-4-methyl-6-[2-methyl-3-[1,3,3,3-tetramethyl-1 -[(trimetthyl-silyl)oxy]disiloxanyl]propyl]phenol (CAS No.: 155633-54-8) having the INCI name Drometrizole Trisiloxane, which is characterized by the chemical structural formula

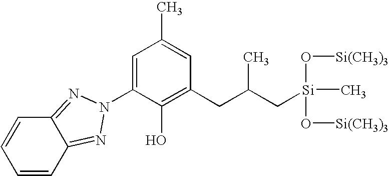

The UV-B and/or broadband filters can be oil-soluble or water-soluble. Examples of advantageous oil-soluble UV-B and/or broadband filter substances are:

3-benzylidenecamphor derivatives, preferably 3-(4-methylbenzylidene)camphor, 3-benzylidenecamphor;

4-aminobenzoic acid derivatives, preferably 2-ethylhexyl 4-(dimethylamino)-benzoate, amyl 4-(dimethylamino)benzoate;

2,4,6-trianilino(p-carbo-2′-ethyl-1′-hexyloxy)-1,3,5-triazine;

esters of benzalmalonic acid, preferably di(2-ethylhexyl) 4-methoxybenzalmalonate,

esters of cinnamic acid, preferably 2-ethylhexyl 4-methoxycinnamate, isopentyl 4-methoxycinnamate;

derivates of benzophenone, preferably 2-hydroxy-4-methoxybenzophenone, 2-hydroxy -4-methoxy-4′-methylbenzophenone, 2,2′-dihydroxy-4-methoxybenzophenone

and UV filters bonded to polymers.

Examples of advantageous water-soluble UV-B and/or broadband filter substances are:

salts of 2-phenylbenzimidazole-5-sulfonic acid, such as its sodium, potassium or its triethanolammonium salt, and also the sulfonic acid itself;

sulfonic acid derivatives of 3-benzylidenecamphor, such as, for example, 4-(2-oxo-3-bornylidenemethyl) benzenesulfonic acid, 2-methyl-5-(2-oxo-3-bornylidenemethyl)-

sulfonic acid and salts thereof.

A further light protection filter substance which can be used advantageously according to the invention is ethylhexyl 2-cyano-3,3-diphenylacrylate (octocrylene), which is available from BASF under the name Uvinul® N 539 and is characterized by the following structure:

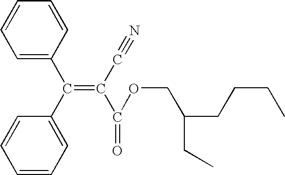

It can also be of considerable advantage to use polymer-bonded or polymeric UV filter substances in the preparations according to the present invention, in particular those described in WO-A-92/20690.

In some instances, it can also be advantageous to incorporate further UV-A and/or UV-B filters in accordance with the invention into cosmetic or dermatological preparations, for example certain salicylic acid derivatives, such as 4-isopropylbenzyl salicylate, 2-ethylhexyl salicylate (=octyl salicylate), homomenthyl salicylate.

The list of given UV filters which can be used for the purposes of the present invention is, of course, not intended to be limiting.

The preparations according to the invention advantageously comprise the substances which absorb UV radiation in the UV-A and/or UV-B region in a total amount of, for example, 0.1% by weight to 30% by weight, preferably 0.5 to 20% by weight, in particular 1.0 to 15.0% by weight, in each case based on the total weight of the preparations, in order to provide cosmetic preparations which protect the hair or the skin from the entire range of ultraviolet radiation. They can also be used as sunscreens for the hair or the skin.

The examples below serve to illustrate the present invention without limiting it. Unless stated otherwise, all amounts, proportions and percentages are based on the weight and the total amount or on the total weight of the preparations.

| Emulsion I | % by wt. |
|---|---|
| Stearic acid | 3.00 |
| Cetyl alcohol | 8.50 |
| PEG-20 stearate¹ | 8.50 |
| Cyclomethicone² | 10.00 |
| c12-13-alkyl lactate | 1.00 |
| Isohexadecane³ | 2.00 |
| Glycerol | 5.00 |
| Sodium hydroxide | q.s. |
| Preservative | q.s. |
| Perfume | q.s. |
| Water, demineralized | ad 100 |
| pH adjusted to 6.5-7.5 | |

To prepare the foam, 90% by volume of emulsion I are foamed with 10%
by volume of a propellant gas mixture of propane and butane. The
foaming can take place, for example by bubbling gas into the preparations
or (vigorously) beating, shaking, spraying or stirring it in the gas
atmosphere in question.
¹Myrj 49, ICI Surfactants
²Dow Corning Fluid 245, Dow Corning
³Solvent ICH, EC Erdolchemie Bayer AG

### EXAMPLES

Example 1

### Foam-like O/W Cream

### Example 2

### O/W Lotion

| Emulsion II | % by wt. |
|---|---|
| Stearic acid | 2.00 |
| Myristyl alcohol | 1.50 |
| Cetylstearyl alcohol | 0.50 |
| PEG-100 stearate¹ | 3.0 |
| Hydrogenated polyisobutene² | 5.0 |
| Cyclomethicone³ | 15.00 |
| Glycerol | 3.00 |
| Sodium hydroxide | q.s. |
| Preservative | q.s. |
| Perfume | q.s. |
| Water, demineralized | ad 100 |
| pH adjusted to 5.0-6.5 | |

To prepare the foam, 70% by volume of emulsion II are foamed with 20%
by volume of carbon dioxide^{a}.
¹Myrj 59p, ICI Surfactants,
²Polysynlan, Chemische Fabrik Lehrte,
³Dow Corning Fluid 245, Dow Corning

| Emulsion III | % by wt. |
|---|---|
| Stearic acid | 5.00 |
| Cetylstearyl alcohol | 5.50 |
| PEG-30 stearate¹ | 1.00 |
| Cyclomethicone² | 10.00 |
| Dimethicone³ | 3.00 |
| Hydrogenated polyisobutene⁴ | 5.00 |
| Citric acid | 0.10 |
| Glycerol | 3.00 |
| Perfume, preservative, | q.s. |
| Sodium hydroxide | q.s. |
| Dyes etc. | q.s. |
| Water | ad 100 |
| pH adjusted to 6.0-7.5 | |

To prepare the foam, 95% by volume of Emulsion III are foamed with 5%
by volume of a propellant gas mixture of propane and butane^{a}.
¹Myrj 51, ICI Surfactants,
²Dow Corning Fluid 245, Dow Corning,
³Wacker silicone oil AK 35, Wacker,
⁴Polysynlan, Chemische Fabrik Lehrte,

### Example 3

### O/W Lotion

| Emulsion IV | % by wt. |
|---|---|
| Palmitic acid | 2.00 |
| Cetyl alcohol | 2.00 |
| PEG-100 stearate¹ | 2.00 |
| Dimethicone² | 2.50 |
| Cyclomethicone³ | 9.50 |
| Dicaprylyl ether⁴ | 2.00 |
| Glycerol | 3.00 |
| Mica | 1.00 |
| Iron oxide | 1.00 |
| Titanium dioxide | 4.50 |
| Vitamin A palmitate | 0.10 |
| Sodium hydroxide | q.s. |
| Preservative | q.s. |
| Perfume | q.s. |
| Water, demineralized | ad 100 |
| pH adjusted to 6.0-7.5 | |

To prepare the foam, 85% by volume of Emulsion IV are foamed with
15% by volume of nitrogen^{a}.
¹Myrj 59p, ICI Surfactants,
²Wacker silicone oil AK 35, Wacker.
³Dow Corning Fluid 245, Dow Corning,
⁴Cetiol OE, Henkel Cognis

### Example 4

### O/W Emulsion Make-up

| Emulsion V | % by wt. |
|---|---|
| Stearic acid | 4.00 |
| Cetyl alcohol | 2.00 |
| PEG-30 stearate¹ | 2.00 |
| Sorbitan monostearate² | 1.50 |
| Propylene glycol monoisostearate | 1.00 |
| Cyclomethicone | 5.00 |
| Dimethicone | 1.00 |
| Vitamin E acetate | 1.00 |
| Retinyl palmitate | 0.20 |
| Glycerol | 3.00 |
| BHT | 0.02 |
| Disodium EDTA | 0.10 |
| Perfume, preservative, dyes | q.s. |
| Potassium hydroxide | q.s. |
| Water, | ad 100 |
| pH adjusted to 5.0-7.0 | |

To prepare the foam, 89% by volume of Emulsion V are foamed with
11% by volume of nitrous oxide^{a}.
¹Myrj 51, ICI Surfactants

### Example 5

### O/W Cream

### Example 6

### O/W Lotion

| Emulsion VI | % by wt. |
|---|---|
| Stearic acid | 4.00 |
| Cetylstearyl alcohol | 1.00 |
| PEG-100 stearate¹ | 1.00 |
| Propylene glycol dicaprylate/ | 3.50 |
| dicaprate | |
| Dimethicone | 0.50 |
| Cyclomethicone | 15.00 |
| Vitamin E acetate | 2.00 |
| Glycerol | 3.00 |
| Perfume, preservative, dyes etc. | q.s. |
| Sodium hydroxide | q.s. |
| Water | ad 100 |
| pH adjusted to 6.0-7.5 | |

To prepare the foam, 92% by volume of Emulsion VI are foamed with
8% by volume of a propellant mixture of propane and butane^{a}.
¹Myrj 59p, ICI Surfactants

| Emulsion VII | % by wt. |
|---|---|
| Stearic acid | 1.00 |
| Cetylstearyl alcohol | 4.00 |
| Myristyl alcohol | 1.00 |
| PEG-20 stearate¹ | 1.00 |
| Caster oil | 2.00 |
| Cyclomethicone | 16.0 |
| Dimethicone | 3.00 |
| Phenyltrimethicone | 1.00 |
| Glycerol | 3.00 |
| Octyl methoxycinnamate² | 4.00 |
| Butylmethoxydibenzoylmethane³ | 3.00 |
| Ethylhexyltriazone⁴ | 3.00 |
| BHT | 0.02 |
| Disodium EDTA | 0.10 |
| Perfume, preservative, dyes, etc. | q.s. |
| Potassium hydroxide | q.s. |
| Water | ad 100 |
| pH adjusted to 5.0-6.0 | |

To prepare the foam, 85% by volume of Emulsion VII are foamed with
15% by volume of helium^{a}
¹Myrj 49, ICI Surfactants,
²Escalol 5571, ISP-Van Dyke,
³Parsol 1789, Hoffmann La Roche,
⁴Uvinul T150

### Example 7

### Sunscreen Cream

That which is claimed:

1. A foamable cosmetic or dermatological preparation comprising:
I. an emulsifier system comprising:
A. at least one emulsifier A selected from the group consisting of wholly neutralized, partially neutralized and unneutralized, branched and unbranched, saturated and unsaturated fatty acids having a chain length of from 10 to 40 carbon atoms,
B. at least one emulsifier B selected from the group consisting of polyethoxylated fatty acid esters having a chain length of from 10 to 40 carbon atoms and a degree of ethoxylation of from 5 to 100, and
C. at least one coemulsifier C selected from the group consisting of saturated and unsaturated, branched and unbranched fatty alcohols having a chain length of from 10 to 40 carbon atoms; and
II. up to 50% by weight—based on the total weight of the foamable preparation—of a lipid phase which comprises one or more lipids selected from the group consisting of silicone oils and silicone waxes.

2. The preparation as claimed in claim 1, wherein the lipid phase comprises up to 40% by weight—based on the total weight of the lipid phase—of additional lipids.

3. The preparation as claimed in claim 1, wherein the lipid phase comprises lipids having different polarities.

4. The preparation as claimed in claim 1, wherein the weight ratio of emulsifier A to emulsifier B to coemulsifier C (A:B:C) is a:b:c, where a, b and c, independently of one another, are from 1 to 5.

5. The preparation as claimed in claim 1, wherein the weight ratio of emulsifier A to emulsifier B to coemulsifier C (A:B:C) is a:b:c, where a, b and c, independently of one another, are from 1 to 3.

6. The preparation as claimed in claim 1, wherein the weight ratio of emulsifier A to emulsifier B to coemulsifier C (A:B:C) is 1:1:1.

7. The preparation as claimed in claim 1, wherein the total amount of the emulsifier A, the emulsifier B and the coemulsifier C is from 1 to 20% by weight, based on the total weight of the preparation.

8. The preparation as claimed in claim 1, wherein the emulsifier system further comprises at least one hydrophilic emulsifier.

9. The preparation as claimed in claim 8, wherein the hydrophilic emulsifier is selected from the group consisting of mono-, di- and tri-fatty acid esters of sorbitol.

10. The preparation as claimed in claim 8, wherein the total amount of the hydrophilic emulsifiers is less than 5% by weight, based on the total weight of the preparation.

11. The preparation as claimed in claim 1, further comprises at least one moisturizer.

12. The preparation as claimed in claim 1, wherein the emulsifier system consists essentially of the emulsifier A, the emulsifier B and the coemulsifier C.

13. A method of producing a foamable cosmetic or dermatological preparation, comprising combining a gaseous ingredient with a cosmetic or dermatological base comprising:
I. an emulsifier system comprising:
A. at least one emulsifier A selected from the group consisting of wholly neutralized, partially neutralized and unneutralized, branched and unbranched, saturated and unsaturated fatty acids having a chain length of from 10 to 40 carbon atoms,
B. at least one emulsifier B selected from the group consisting of polyethoxylated fatty acid esters having a chain length of from 10 to 40 carbon atoms and a degree of ethoxylation of from 5 to 100, and
C. at least one coemulsifier C selected from the group consisting of saturated and unsaturated, branched and unbranched fatty alcohols having a chain length of from 10 to 40 carbon atoms; and
II. up to 50% by weight—based on the total weight of the foamable preparation—of a lipid phase which comprises one or more lipids selected from the group consisting of silicone oils and silicone waxes.

14. The method as claimed in claim 13, wherein said gaseous ingredient and said cosmetic or dermatological bases are combined in a pressurized gas container.

15. The method as claimed in claim 14, wherein the gaseous ingredient is selected from the group consisting of linear and branched, halogenated and nonhalogenated hydrocarbons; carbon dioxide; oxygen; compressed air; nitrogen and dimethyl ether.

16. The method as claimed in claim 14, wherein the pressurized gas container is in the form of a cylindrical vessel.

17. The method as claimed in claim 14, wherein the pressurized gas container is formed of metal, shatterproof glass or plastic.

18. The method as claimed in claim 14, wherein the pressurized gas container is formed of protected, shattering glass or protected, shattering plastic.

19. The method as claimed in claim 13, further comprising the step of producing a foam from the foamable cosmetic or dermatological preparation.

20. The method as claimed in claim 19, wherein said foam is produced upon removal from a pressurized gas container.
